(19) United States
(12) Patent Application Publication
(10) Pub. No.: US 2010/0228330 A1
(43) Pub. Date: Sep. 9, 2010

(54) LEAD CONFIGURED FOR HISIAN, PARA-HISIAN, RV SEPTUM AND RV OUTFLOW TRACT PACING

(75) Inventor: Gene A. Bornzin, Simi Valley, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/398,963

(22) Filed: Mar. 5, 2009

(51) Int. Cl. A61N 1/362

(52) U.S. Cl. 607/122

(57) ABSTRACT

Disclosed herein is an implantable medical lead for implantation within a right ventricle of a heart and powered by an implantable pulse generator. The lead includes a lead body having a proximal end configured to couple to the generator, a distal end, an electrode at the distal end, and a distal portion extending proximally from the distal end. When the distal portion is in a non-deflected state, the distal portion biases to assume a configuration including first, second and third generally straight segments and first and second bends. The first segment is proximal of the distal end, the second segment is proximal of the first segment, the third segment is proximal of the second segment, the first bend is between the first and second segments, and the second bend is between the second and third segments. When the distal portion is implanted in the right ventricle, the configuration is at least partially the cause of the electrode being at least one of: positioned against the right ventricle septum; positioned in the outflow tract of the right ventricle; positioned for Hisian pacing; and positioned for para-Hisian pacing.

### FIELD OF THE INVENTION

The present invention relates to medical apparatus and methods. More specifically, the present invention relates to implantable cardiac electrotherapy leads and delivery tools for and methods of using such leads.

### BACKGROUND

Right ventricle (“RV”) septal pacing and Hisian pacing have been shown to be hemodynamically superior to RV apical pacing. For example, when the lead pacing electrode is placed precisely in the proximity of the His region, the surface QRS complex matches the intrinsic conducted R-wave. Also, in patients with a healthy His-purkinje system, the sequence of ventricular activation matches the intrinsic activation.

At least one trial has demonstrated that a high degree of ventricular pacing in the DDD mode was associated with double the likelihood of hospitalization for congestive heart failure (“CHF”) or death when compared to VVI backup pacing. Because of this observation, most clinicians try to avoid ventricular apical pacing by programming a prolonged atrial-ventriclular (“AV”) delay as long as 400 ms. Many of these patients are on amiodarone, calcium channel blockers, digitalis, and/or beta blocking drugs. These drugs, alone or in combination, prolong AV conduction, resulting in a pharmaceutically induced first order heart block. In this situation, the short interval between the previous ventricular systolic event and the atrial contraction leads to insufficient ventricular filling and contributes to mitral regurgitation in mid or late diastole. Although these patients may benefit from restoration of an appropriate AV delay, the fear that ventricular pacing will worsen their condition precludes restoration of AV synchrony. If these patients were provided with Hisian pacing, both the atrial contribution and the optimal sequence of activation would allow for maintenance of optimal cardiac function.

There is a population of symptomatic atrial fibrillation (“AF”) patients that would benefit from the “ablate and pace” therapy option. If these patients received His pacing, their hemodynamic function would be preserved and they would fair better than those patients relegated to RV pacing.

As can be understood from the preceding discussion, Hisian and RV septal pacing offer benefits for a variety of patient conditions. Accordingly, implanting clinicians are generally receptive to the concept of Hisian and septal pacing for a variety of patients, including brady patients, implantable cardioverter defibrillator (“ICD”) patients or, essentially, any dual chamber pacemaker patient. Unfortunately, the ability to reliably deliver Hisian, para-Hisian, RV septal or outflow tract pacing has been elusive.

There is a need in the art for a lead configured to facilitate reliable delivery of Hisian, para-Hisian, outflow tract and RV septal pacing. There is also a need in the art for a method of reliably delivering Hisian, para-Hisian, outflow tract and RV septal pacing.

### BRIEF SUMMARY

Disclosed herein is an implantable medical lead for implantation within a right ventricle of a heart and powered by an implantable pulse generator. In one embodiment, the lead includes a lead body having a proximal end configured to couple to the generator, a distal end, an electrode at the distal end, and a distal portion extending proximally from the distal end. When the distal portion is in a non-deflected state, the distal portion biases to assume a configuration including first, second and third generally straight segments and first and second bends. The first segment is proximal of the distal end, the second segment is proximal of the first segment, the third segment is proximal of the second segment, the first bend is between the first and second segments, and the second bend is between the second and third segments. When the distal portion is implanted in the right ventricle, the configuration is at least partially the cause of the electrode being at least one of: positioned against the right ventricle septum; positioned in the outflow tract of the right ventricle; positioned for Hisian pacing; and positioned for para-Hisian pacing.

Disclosed herein is a tool for delivering a distal portion of an implantable medical lead to an implantation location within a right ventricle of a heart. In one embodiment, the tool includes a body including a proximal end engageable to manipulate the tool during lead implantation, a distal end, and a distal portion extending proximally from the distal end. When the distal portion is in a non-deflected state, the distal portion biases to assume a configuration including first, second and third generally straight segments and first and second bends. The first segment is proximal of the distal end, the second segment is proximal of the first segment, the third segment is proximal of the second segment, the first bend is between the first and second segments, and the second bend is between the second and third segments. When the distal portion is located in the right ventricle, the configuration is at least partially the cause of the distal end being at least one of positioned near the right ventricle septum and positioned in the outflow tract of the right ventricle.

Disclosed herein is a method of implanting an implantable medical lead in a right ventricle of a heart. In one embodiment, the method includes providing a lead body including a proximal end configured to couple to an implantable pulse generator, a distal end, an electrode at the distal end, and a distal portion extending proximally from the distal end. When the distal portion is in a non-deflected state, the distal portion biases to assume a configuration including first, second and third generally straight segments and first and second bends. The first segment is proximal of the distal end, the second segment is proximal of the first segment, the third segment is proximal of the second segment, the first bend is between the first and second segments, and the second bend is between the second and third segments. The method further includes: deflecting the distal portion out of its non-deflected state to deliver the distal portion into the right ventricle; and allowing the distal portion to assume its non-deflected state within the right ventricle, wherein the configuration is at least partially the cause of the electrode being at least one of: positioned against the right ventricle septum; positioned in the outflow tract of the right ventricle; positioned for Hisian pacing; and positioned for para-Hisian pacing.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 depicts an implantable cardiac electrotherapy lead deployed in the heart, wherein a distal portion of the lead is formed to bias into a shape that facilitates Hisian, para-Hisian, right ventricular septum or outflow tract pacing.

FIG. 2 is the lead of FIG. 1 shown by itself for clarity purposes.

FIG. 3A depicts the lead of FIG. 2, wherein the preformed bends are in different planes.

FIG. 3B depicts the angles between the relatively straight portions of the lead of FIG. 2.

FIG. 4A depicts a guidewire with preformed bends in different planes.

FIG. 4B is the guidewire of FIG. 4A, wherein the angles between the relatively straight portions of the guidewire are shown.

FIG. 5A depicts a stylet with preformed bends in different planes.

FIG. 5B is the stylet of FIG. 5A, wherein the angles between the relatively straight portions of the stylet are shown.

FIG. 6A depicts an introducer catheter or sheath with preformed bends in different planes.

FIG. 6B is the introducer catheter or sheath of FIG. 6A, wherein the angles between the relatively straight portions of the introducer catheter or sheath are shown.

### DETAILED DESCRIPTION

The present application describes an implantable cardiac electrotherapy lead 5, such as an active fixation bradycardia or tachycardia lead. The lead 5 includes a distal portion 7 that is configured or formed to bias into a shape that facilitates Hisian, para-Hisian, right ventricular (“RV”) septum or outflow tract pacing.

In one embodiment, the distal portion 7 of the lead 5 has a first bend 16 and a second bend 18 in different planes. Because of the shape of its distal portion 7, the lead 5 may be placed more easily and with overall higher stability in the ventricular septum. Also, there is a reduced likelihood of cardiac tissue perforation and dislodgement of the lead distal end 20 due to the placement of the lead distal end in the outflow tract. In addition, a longer shocking coil 75 may be used, which may reduce the defibrillation threshold (“DFT”). The lead 5 may provide improved hemodynamics through the sequence of activation optimization via RV septal pacing.

The present application also describes delivery tools such as a guidewire 95, a stylet 115 and an introducer sheath 135 to facilitate delivery of the lead 5. In some embodiments, the distal portions of these delivery tools 95, 115, 135 are configured similar to the distal portion 7 of the lead 5 to facilitate delivery of the lead distal end to the appropriate implant site for Hisian, para-Hisian, outflow tract or RV septum pacing.

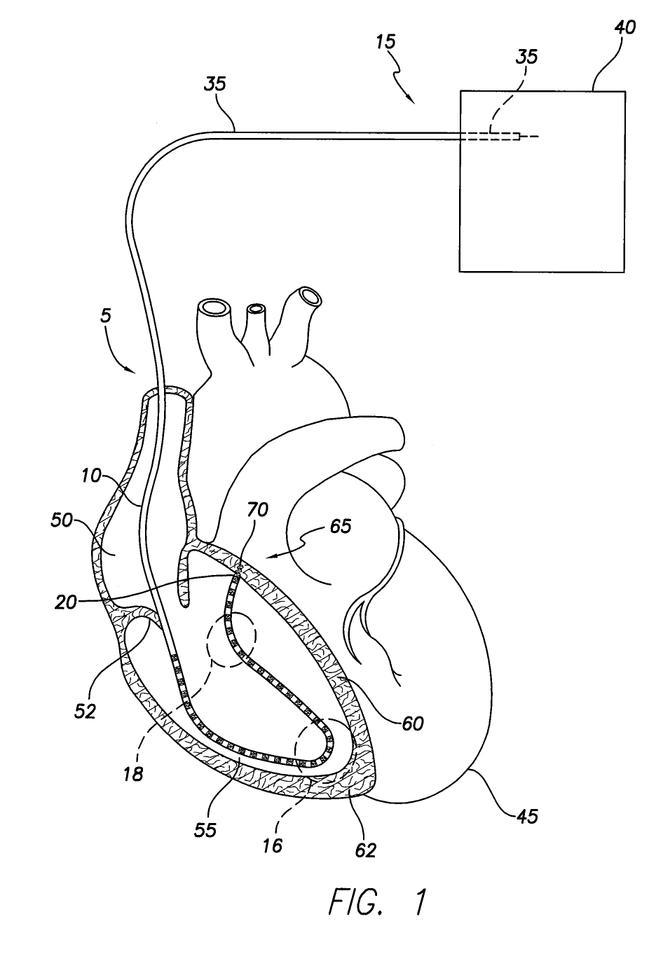

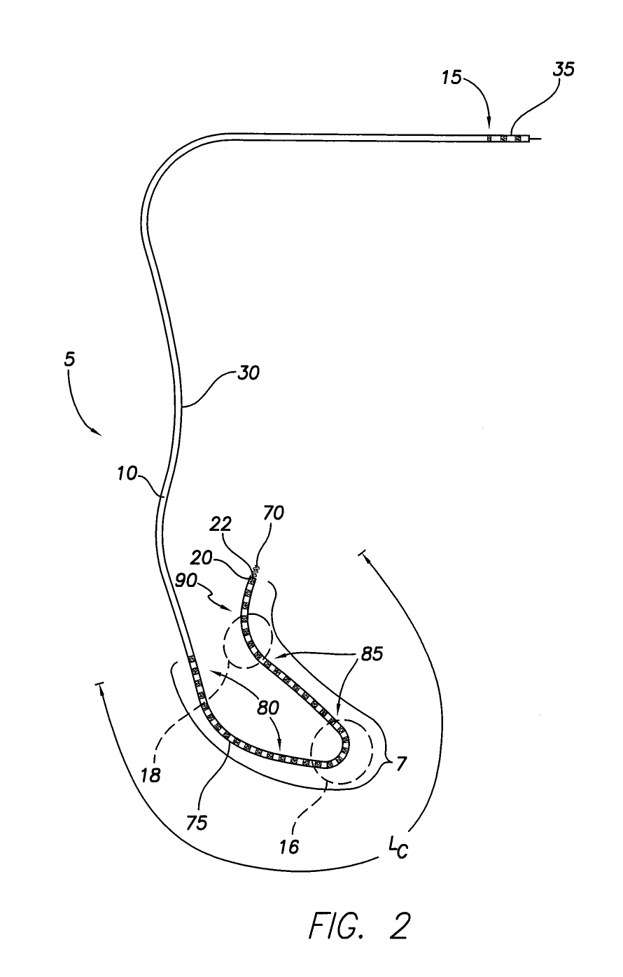

For a discussion of an embodiment of the implantable cardiac electrotherapy lead 5, reference is made to FIGS. 1 and 2. FIG. 1 depicts the implantable cardiac electrotherapy lead 5 deployed in the heart 45, wherein a distal portion 7 of the lead 5 is formed to bias into a shape that facilitates Hisian, para-Hisian, right ventricular septum or outflow tract pacing. FIG. 2 is the lead 5 of FIG. 1, shown by itself for clarity purposes.

As shown in FIGS. 1 and 2, the preformed lead 5 has a lead tubular body 10 with a proximal end 15, two preformed bends 16, 18, a distal end 20 and a tubular sheath or housing 25 extending between the ends 15, 20. The two bends 16, 18 and the distal end 20 form the distal portion 7 of the lead, which is configured to place the distal end 20 at the proper spot for Hisian, para-Hisian, outflow tract or RV septum pacing. The tubular housing 25 is made of an insulating, biocompatible, biostable material such as silicone rubber, polyurethane, or a copolymer (e.g., silicone rubber-polyurethane-copolymer (“SPC”)). The tubular housing 25 defines an outer circumferential surface 30 of the lead 5.

As indicated in FIG. 1, the proximal end 15 of the lead body 10 incorporates a connector assembly 35 for connecting the lead body 10 to a pulse generator 40 such as a pacemaker, defibrillator or implantable cardioverter defibrillator (“ICD”). The connector assembly 35 of the lead 5 is received within a receptacle of the pulse generator 40, where seals prevent the ingress of body fluids into the receptacle.

As illustrated in FIG. 1, when the lead 5 is deployed in the heart 45, the lead body 10 extends through the right atrium (“RA”) 50 and into the right ventricle (“RV”) 55, with the lead distal end 20 affixed at the RV septum 60 of the heart 45. A proximal or first bend 16 is located near the ventricular apex 62 and a distal or second bend 18 is located near the RV septum 60 and the outflow tract 65.

In one embodiment, as depicted in FIG. 2, the distal end 20 includes a distal tip that terminates at a distal end face 22, which forms the extreme distal end of the lead 5. The distal end face 22 defines a central aperture through which an anchor 70 may be extended to anchor the lead distal end 15 to the myocardial tissue, and more specifically, the RV septum near or in the outflow tract and/or at or near the bundle of His. The anchor 70 may be a pacing and/or sensing electrode for serving as the distal tip electrode of the lead. In another embodiment, the shape of the lead distal portion 7 causes the lead body 10 to bias against the walls of the RV, thereby providing a passive fixation means to maintain the lead distal end 20 at the proper pacing site. In such an embodiment, the electrode 70 may not be an anchor type electrode, but may be simply an atraumatic distal tip electrode 70 for pacing and/or sensing, as commonly found on passive pacing leads.

As depicted in FIGS. 1 and 2, in some embodiments, the lead body 10 may further carry a cardioverting-defibrillating electrode 75, which may be in the form of an elongated coil wound about the outer circumferential surface 30 of the insulating housing 25. In one embodiment, when the lead 5 is implanted in the heart 45, the proximal end of the electrode 75 is approximately adjacent to the tricuspid valve 52 and the distal end of electrode 75 is approximately at the distal end 20 of lead 5. In one embodiment, the elongated electrode coil 75 may have a length L_{c} of approximately 8.5 cm. In one embodiment, the elongated electrode coil 75 may have a length L_{c} of between approximately 5.5 cm and approximately 12 cm.

The elongated coil 75 is advantageous because the increased length reduces shocking impedance as compared to standard shocking coils. Accordingly, the elongated coil 75 allows for a higher current flux density in the ventricles during the early phase of shock delivery. This has an effect of DFT, a benefit of which is higher confidence in defibrillating patients, such as patients with “baggy” hearts.

When the lead distal portion 7 is generally free from exterior forces (e.g., the lead distal portion 7 is not being deflected by a delivery tool or the walls of a vascular system or structure), the lead distal portion 7 may bias into a configuration similar to that depicted in FIG. 2. Thus, as shown in FIG. 2, the distal portion 7 of the lead 5, when in a non-deflected state, has two preformed bends 16, 18. The lead body 10 extends distally along a proximal or first generally straight portion 80 from the proximal end 15 of the lead 5 to a first bend 16. From the proximal or first bend 16, the lead body 10 extends distally along a middle generally straight portion 85 to a distal or second bend 18. From the second bend 18, the lead body 10 extends distally along a distal or third generally straight portion 90 to the distal end 20 of the lead 5.

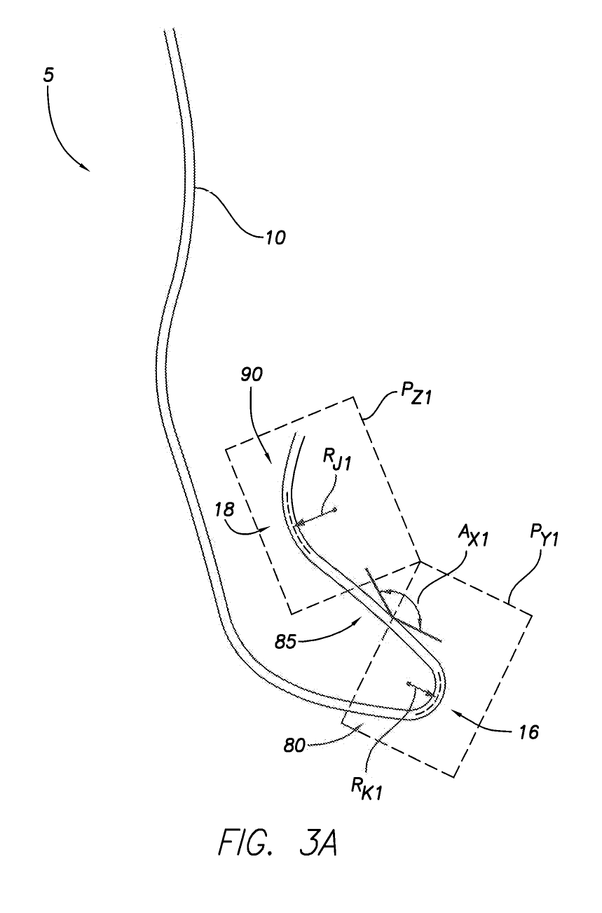

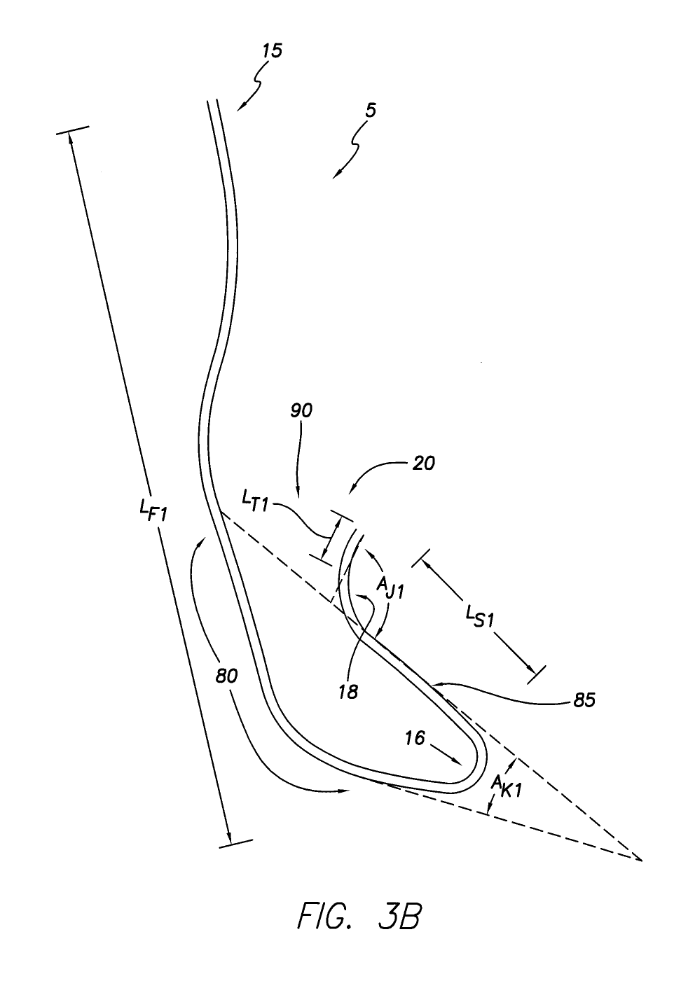

For a more detailed discussion of the configuration of the lead distal portion 7, reference is now made to FIGS. 3A and 3B. FIG. 3A depicts the lead 5 of FIG. 2, wherein the preformed bends 16, 18 are in different planes P_{Y1}, P_{Z1}. FIG. 3B depicts the angles A_{J1} and A_{K1} between the generally straight portions 80, 85, 90 of the lead 5 of FIG. 2.

As illustrated in FIG. 3A, the lead 5 has two preformed bends 16, 18 on different planes P_{Y1}, P_{Z1}. In other words, in one embodiment, the first or proximal bend 16 exists in a different plane from the second or distal bend 18. Between the two planes P_{Y1}, P_{Z1} is an angle A_{X1} of between approximately 60 degrees and approximately 120 degrees. In one embodiment, the angle A_{X1} may be approximately 90 degrees. The first bend 16 has a radius R_{K1} for a centerline of the lead body 10 of between approximately 2 cm and approximately 4.25 cm. In one embodiment the radius R_{K1} may be approximately 3 cm. The second bend 18 has a radius R_{J1} for a centerline of the lead body 10 of between approximately 1.5 cm and approximately 4 cm. In one embodiment the radius R_{J1} may be approximately 2 cm.

As shown in FIG. 3B, the lead 5 has three generally straight portions 80, 85, 90 and two preformed bends 16, 18. The first or most proximal generally straight portion 80 has a length L_{F1} that extends proximally to the proximal end 15 of the measurement (which may be at the connector extension that may be trifurcated or bifurcated) from the proximal bend 16 and may be between approximately 23 cm and approximately 52 cm. In one embodiment, the length L_{F1} may be approximately 33 cm. The middle or second generally straight portion 85 has a length L_{S1} that extends between the proximal and distal bends 16, 18 and may be between approximately 2 cm and approximately 5.5 cm. In one embodiment, the length L_{S1} may be approximately 3.5 cm. The distal or third generally straight portion 90 has a length L_{T1} that extends distally to the distal end 20 from the distal bend 18 and may be between approximately 1 cm and approximately 6 cm. In one embodiment, the length L_{T1} may be approximately 3.5 cm. The proximal or first bend 16 is also defined by the angle A_{K1} created between the proximal generally straight portion 80 and the middle generally straight portion 85. The angle A_{K1} is between approximately zero degrees and approximately 35 degrees. In one embodiment, the angle A_{K1} may be approximately 20 degrees. The distal or second bend 18 is also defined by the angle A_{J1} created between the middle generally straight portion 85 and the distal generally straight portion 90. The angle A_{J1} is between approximately 30 degrees and approximately 70 degrees. In one embodiment, the angle A_{J1} may be approximately 45 degrees.

It should be noted that while the proximal or first generally straight portion 80 is depicted in FIGS. 1-3B as having some bend, the lead body 10 is, of course, quite “floppy” or pliable and will be generally straight unless deflected by an outside force or laid out in a curved manner. The proximal straight portion 80 does not include preformed curved portions like the bends 16, 18 separating the straight portions 80, 85, 90 and which bias into the curved configurations depicted in FIGS. 1-3B, unless acted on by an outside force to be another shape. In other words, as can be understood from FIG. 2, when the distal portion of the lead body (i.e., the portion of the lead body having the three generally straight segments 80, 85, 90 and the two bends 16, 18) is in a non-deflected state (i.e., not acted on by forces that are not part of the lead body itself), the distal portion biases to assume a configuration including three generally straight segments 80, 85, 90 and the two bends 16, 18. Thus, as shown in FIG. 1, when the distal portion is implanted in the right ventricle, the configuration causes the electrode 70 to be at least one of: positioned against the right ventricle septum; positioned in the right ventricle outflow tract; positioned for Hisian pacing; and positioned for para-Hisian pacing.

The distal portion 7 of the lead 5 can be preformed to have the bends 16, 18 by heat treating SPC or by pre-shaping silicone rubber in the “green state” and finalizing the cure with the prescribed shape. Alternatively or additionally, the shock coil 75 can be heat treated for the bends 16, 18 to provide the lead distal portion 7 with the configuration depicted in FIGS. 1-3B.

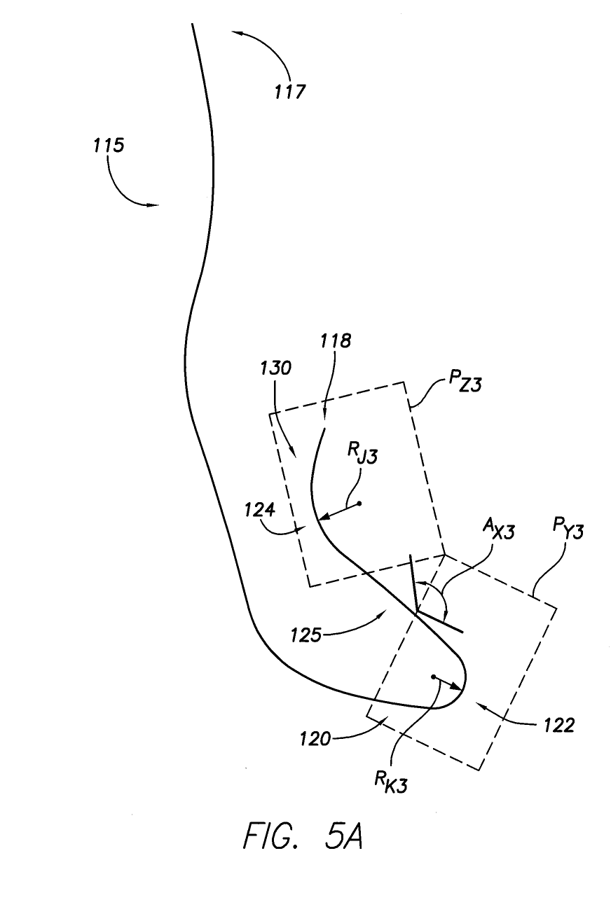

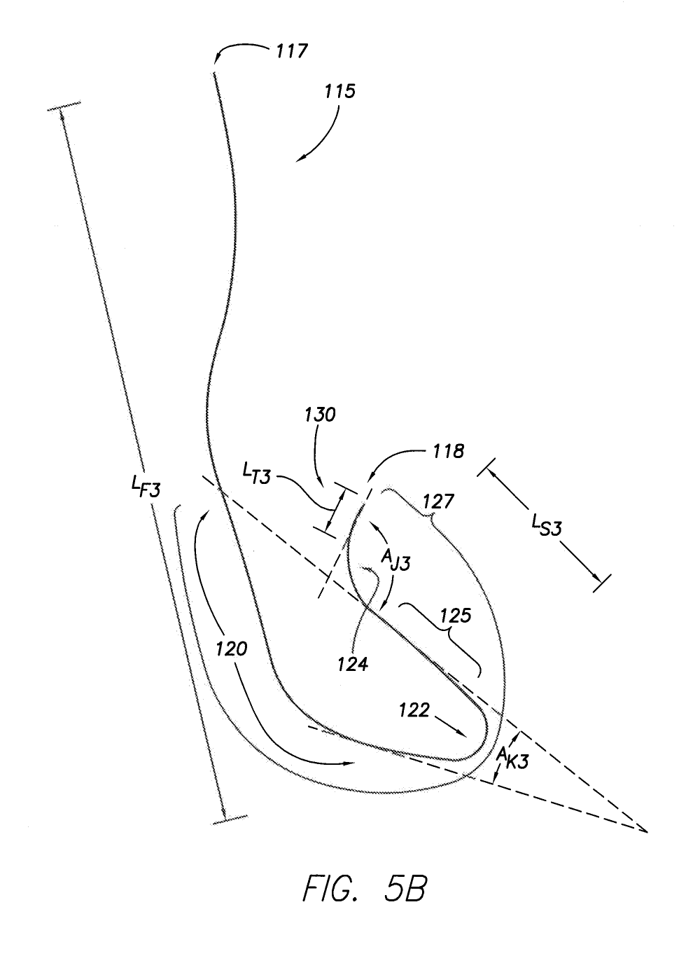

In one alternative embodiment, the lead distal portion 7 is essentially configured as that of a common lead that is not pre-shaped. Such a lead, once implanted or during the implantation process, has a pre-shaped member inserted into a lumen in the lead body, the pre-shaped member being shaped as discussed below with respect to the stylet 115 of FIGS. 5A and 5B. The pre-shaped member (e.g., stylet 115) is left in the implanted lead to cause the lead to remain in the biased shape discussed with respect to FIGS. 1-3B.

A lead 5 configured as described above with respect to FIGS. 1-3B is advantageous for several reasons, including but not limited to, because the configuration allows the lead 5 to pace the RV septum (and, in one embodiment, to or near the bundle of His) and stabilize the pacing electrode in this location. Additionally, the shape of the lead 5 will generally help direct the lead distal tip anchor electrode 70 to the desired site in the RV septum (e.g., at or near the location of the bundle of His) and may help it to stay in place. Further, a lead placed in the outflow tract does not tend to perforate and tamponade the RV.

As discussed previously, the delivery tools 95, 115, 135 may be configured similar to the distal portion 7 of the lead 5 to facilitate delivery of the lead distal end 20 (and, more specifically, the distal tip electrode 20) to the appropriate implant site (e.g., at or near the location of the bundle of His in the RV septum at or near the outflow tract). In other words, the distal portion of the delivery tools 95, 115, 135 is formed to bias into a shape that facilitates delivery of the lead distal end electrode 70 to an implant location that facilitates Hisian, para-Hisian, RV and outflow tract pacing.

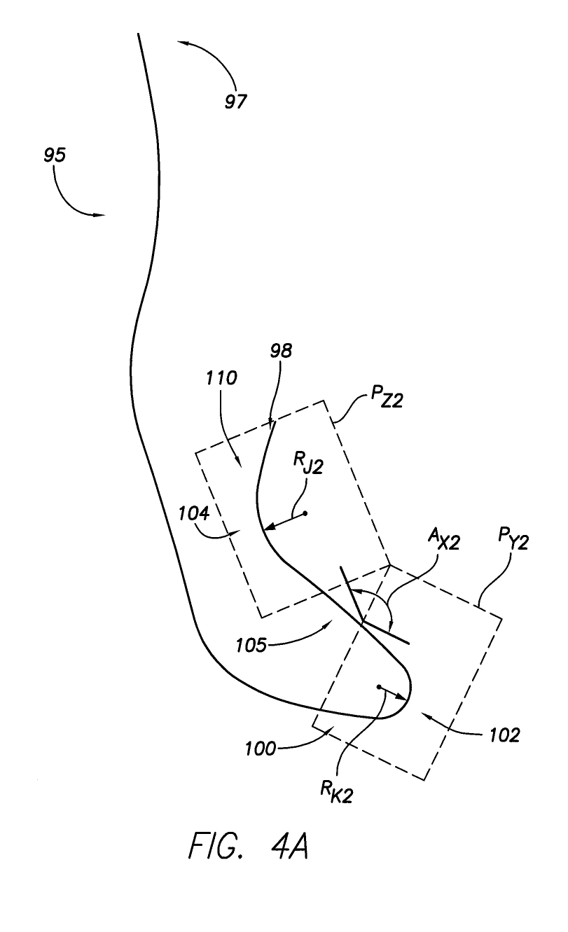

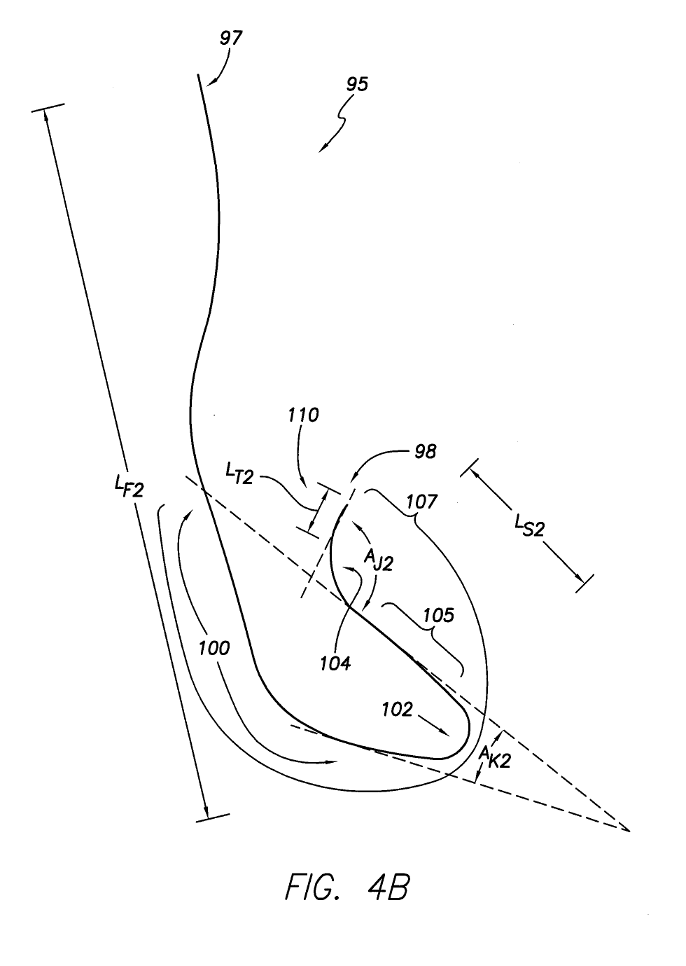

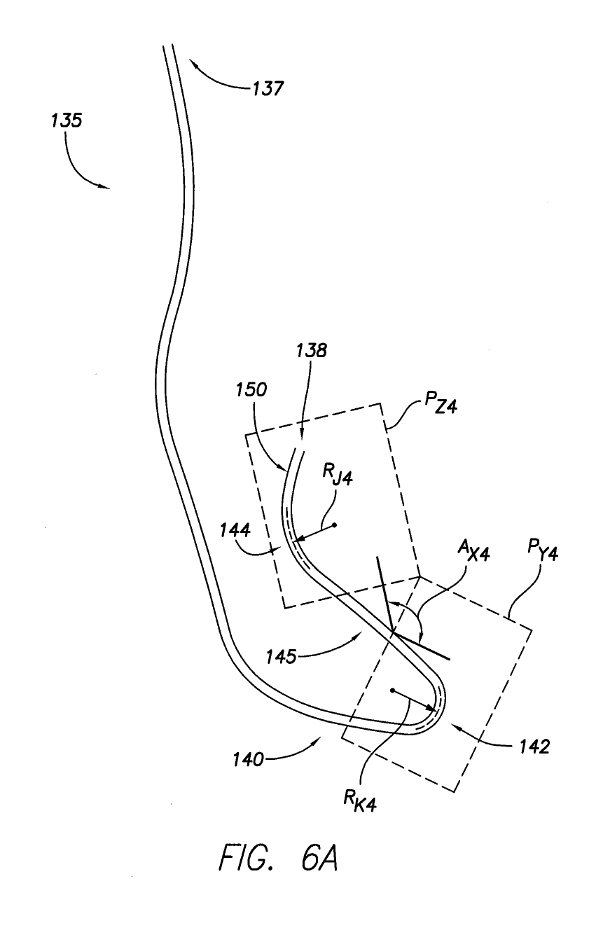

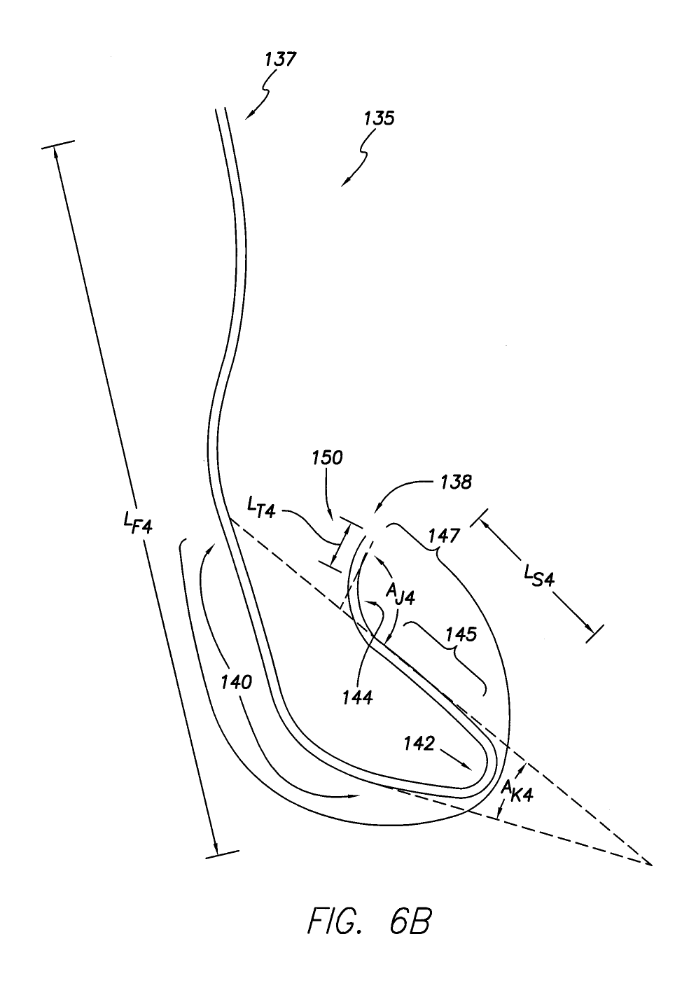

For a more detailed discussion of delivery tools that may be utilized with the lead 5, reference is now made to FIGS. 4A, 4B, 5A, 5B, 6A and 6B. FIG. 4A depicts a guidewire 95 with preformed bends 102,104 in different planes P_{Y2}, P_{Z2}. FIG. 4B is the guidewire 95 of FIG. 4A, wherein the angles A_{J2} and A_{K2} between the relatively straight portions 100, 105, 110 of the guidewire 95 are shown. FIG. 5A depicts a stylet 115 with preformed bends 122, 124 in different planes P_{Y3}, P_{Z3}. FIG. 5B is the stylet 115 of FIG. 5A, wherein the angles A_{J3} and A_{K3} between the relatively straight portions 120, 125, 130 of the stylet 115 are shown. FIG. 6A depicts an introducer catheter or sheath 135 with preformed bends 142,144 in different planes P_{Y4}, P_{Z4}. FIG. 6B is the introducer catheter or sheath 135 of FIG. 6A, wherein the angles A_{J4} and A_{K4} between the relatively straight portions 140, 145, 150 of the introducer catheter or sheath 135 are shown.

As can be understood with reference to FIGS. 1, 4A, 4B, 5A, 5B, 6A and 6B, the guidewire 95, stylet 115 and introducer sheath or catheter 135 can be used as delivery tools and/or to facilitate placement of the lead 5. As discussed in more detail above, the lead 5 has a lead tubular body 10 with a proximal end 15, a distal portion 7, preformed bends 16, 18, a distal end 20 and a tubular sheath or housing 25 extending between the ends 15, 20. The tubular sheath or housing defines a lumen through which the guidewire 95 or stylet 115 may be extended. Passage of the guidewire 95 or stylet 115 through the lumen of the lead 5 assists in guiding the lead 5 to its appropriate location in the ventricle, and maintains the lead 5 in position while the anchor 70 is advanced by the stylet 115 into the myocardial tissue, and more specifically into the RV septum 60. The lead 5, stylet 115 and guidewire 105 may access an internal vessel, such as a vein or artery, via the introducer sheath 135, which may be used as an entry or exit portal into the internal vessel.

For a more detailed discussion of the configuration of the distal portion 107 of the guidewire 95, reference is now made to FIGS. 4A-4B. In some embodiments, the guidewire 95 may be hollow. As shown in FIGS. 4A-4B, the distal portion 107 of the guidewire 95, when in a non-deflected state, has two preformed bends 102, 104. The guidewire 95 extends distally along a proximal or first generally straight portion 100 from the proximal end 97 of the guidewire 95 to a first bend 102. From the proximal or first bend 102, the guidewire 95 extends distally along a middle generally straight portion 105 to a distal or second bend 104. From the second bend 104, the guidewire 95 extends distally along a distal or third generally straight portion 110 to the distal end 98 of the guidewire 95.

As illustrated in FIGS. 4A and 4B, the guidewire 95 has two preformed bends 102, 104 on different planes P_{Y2}, P_{Z2}. In other words, in one embodiment, the first or proximal bend 102 exists in a different plane from the second or distal bend 104. Between the two planes P_{Y2}, P_{Z2} is an angle A_{X2} of between approximately 60 degrees and approximately 120 degrees. In one embodiment, the angle A_{X2} may be approximately 90 degrees. The first bend 102 has a radius R_{K2} for a centerline of the guidewire 95 of between approximately 2 cm and approximately 4.25 cm. In one embodiment, the radius R_{K2} may be approximately 3 cm. The second bend 104 has a radius R_{J2} for a centerline of the guidewire 95 of between approximately 1.5 cm and approximately 4 cm. In one embodiment, the radius R_{J2} may be approximately 2 cm.

As shown in FIGS. 4A and 4B, the guidewire 95 has three generally straight portions 100, 105,110 and two preformed bends 102, 104. The first or most proximal generally straight portion 100 has a length L_{F2} that extends proximally from the proximal bend 102 to the proximal end 97and may be between approximately 23 cm and approximately 52 cm. In one embodiment, the length L_{F2} may be approximately 33 cm. The middle or second generally straight portion 105 has a length L_{S2} that extends between the proximal and distal bends 102, 104 and may be between approximately 2 cm and approximately 5.5 cm. In one embodiment, the length L_{S2} may be approximately 3.5 cm. The third or distal generally straight portion 110 has a length L_{T2} that extends distally from the distal bend 104 to the distal end 98 between approximately 1 cm and approximately 6 cm. In one embodiment, the length L_{T2} may be approximately 3.5 cm. The proximal or first bend 102 is also defined by the angle A_{K2} created between the first generally straight portion 100 and the middle generally straight portion 105. The angle A_{K2} is between approximately zero degrees and approximately 35 degrees. In one embodiment, the angle A_{K2} may be approximately 20 degrees. The distal or second bend 104 is also defined by the angle A_{J2} created between the middle generally straight portion 105 and the distal generally straight portion 110. The angle A_{J2} is between approximately 30 degrees and approximately 70 degrees. In one embodiment, the angle A_{J2} may be approximately 45 degrees.

The guidewire 95 can be preformed by bending or etc. The guidewire 95 can have a diameter of approximately 0.15″.

For a more detailed discussion of the configuration of the distal portion 127 of the stylet 115, reference is now made to FIGS. 5A-5B. As shown in FIGS. 5A-5B, the distal portion 127 of the stylet 115 has two preformed bends 122, 124. The stylet 115 extends distally along a proximal or first generally straight portion 120 from the proximal end 117 of the stylet 115 to a first bend 122. From the proximal or first bend 122, the stylet 115 extends distally along a middle generally straight portion 125 to a distal or second bend 124. From the second bend 124, the stylet 115 extends distally along a distal or third generally straight portion 130 to the distal end 118 of the stylet 115.

As illustrated in FIGS. 5A and 5B, the stylet 115 has two preformed bends 122, 124 on different planes P_{Y3}, P_{Z3}. In other words, in one embodiment, the first or proximal bend 122 exists in a different plane from the second or distal bend 124. Between the two planes P_{Y3}, P_{Z3} is an angle A_{X3} of between approximately 60 degrees and approximately 120 degrees. In one embodiment, the angle A_{X3} may be approximately 90 degrees. The first bend 122 has a radius R_{K3} for a centerline of the stylet 115 of between approximately 2 cm and approximately 4.25 cm. In one embodiment, the radius R_{K3} may be approximately 3 cm. The second bend 124 has a radius R_{J3} for a centerline of the stylet 115 of between approximately 1.5 cm and approximately 4 cm. In one embodiment, the radius R_{J3} may be approximately 2 cm.

As shown in FIGS. 5A and 5B, the stylet 115 has three generally straight portions 120, 125,130 and two preformed bends 122, 124. The first or proximal generally straight portion 120 has a length L_{F3} that extends proximally from the proximal bend 122 to the proximal end 117 and may be between approximately 23 cm and approximately 52 cm. In one embodiment, the length L_{F3} may be approximately 33 cm. The middle or second generally straight portion 125 has a length L_{S3} that extends between the proximal and distal bends 122, 124 and may be between approximately 2 cm and approximately 5.5 cm. In one embodiment, the length L_{S3} may be approximately 3.5 cm. The distal or third generally straight portion 130 has a length L_{T3} that extends distally from the distal bend 124 to the distal end 118 and may be between approximately 1 cm and approximately 6 cm. In one embodiment, the length L_{T3} may be approximately 3.5 cm. The proximal or first bend 122 is also defined by the angle A_{K3} created between the proximal generally straight portion 120 and the middle generally straight portion 125. The angle A_{K3} is between approximately zero degrees and approximately 35 degrees. In one embodiment, the angle A_{K3} may be approximately 20 degrees. The distal or second bend 124 is also defined by the angle A_{J3} created between the middle generally straight portion 125 and the distal generally straight portion 130. The angle A_{J3} is between approximately 30 degrees and approximately 70 degrees. In one embodiment, the angle A_{J3} may be approximately 45 degrees.

The stylet 115 can be preformed by bending or etc. The stylet 115 can have a diameter of approximately 0.15″.

For a more detailed discussion of the configuration of the distal portion 137 of the introducer sheath 135, reference is now made to FIGS. 6A-6B. As shown in FIGS. 6A-6B, the distal portion 137 of the introducer sheath 135, when in a non-deflected state, has two preformed bends 142, 144. The introducer sheath 135 extends distally along a proximal or first generally straight portion 140 from the proximal end 137 of the introducer sheath 135 to a first bend 142. From the proximal or first bend 142, the introducer sheath 135 extends distally along a middle generally straight portion 145 to a distal or second bend 144. From the second bend 144, the introducer sheath 135 extends distally along a distal or third generally straight portion 150 to the distal end 138 of the introducer sheath 135.

As illustrated in FIGS. 6A and 6B, the introducer sheath 135 has two preformed bends 142,144 on different planes P_{Y4}, P_{Z4}. In other words, in one embodiment, the first or proximal bend 142 exists in a different plane from the second or distal bend 144. Between the two planes P_{Y4}, P_{Z4} is an angle A_{X4} of between approximately 60 degrees and approximately 120 degrees. In one embodiment, the angle A_{X4} may be approximately 90 degrees. The first bend 142 has a radius R_{K4} for a centerline of the introducer sheath 135 of between approximately 2 cm and approximately 4.25 cm. In one embodiment, the radius R_{K4} may be approximately 3 cm. The second bend 144 has a radius R_{J4} for a centerline of the introducer sheath 135 of between approximately 1.5 cm and approximately 4 cm. In one embodiment, the radius R_{J4} may be approximately 2 cm.

As shown in FIGS. 6A and 6B, the introducer sheath 135 has three generally straight portions 140, 145, 150 and two preformed bends 142,144. The proximal or first generally straight portion 140 has a length L_{F4} that extends proximally from the proximal bend 142 to the proximal end 137 and may be between approximately 23 cm and approximately 52 cm. In one embodiment, the length L_{F4} may be approximately 33 cm. The middle or second generally straight portion 145 has a length L_{S4} that extends between the proximal bend 142 and the distal bend 144 and may be between approximately 2 cm and approximately 5.5 cm. In one embodiment, the length L_{S4} may be approximately 3.5 cm. The third or proximal generally straight portion 150 has a length L_{T4} that extends distally from the distal bend 144 to the distal end 138 and may be between approximately 1 cm and approximately 6 cm. In one embodiment, the length L_{T4} may be approximately 3.5 cm. The proximal or first bend 142 is also defined by the angle A_{K4} created between the proximal generally straight portion 140 and the middle generally straight portion 145. The angle A_{K4} is between approximately zero degrees and approximately 35 degrees. In one embodiment, the angle A_{K4} may be approximately 20 degrees. The distal or second bend 142 is also defined by the angle A_{J4} created between the middle generally straight portion 145 and the distal generally straight portion 150. The angle A_{J4} is between approximately 30 degrees and approximately 70 degrees. In one embodiment, the angle A_{J4} may be approximately 45 degrees.

The introducer sheath 135 can have an outside diameter of between approximately 4F and approximately 9 F.

A benefit of the pre-shaped sheath is it may a lumenless lead to the implantation site. Lumenless leads are leads of a very small diameter that do not have a central lumen.

In one embodiment where the lead 5 is pre-shaped, the lead distal end 20 may be delivered to the implant site via any one or more of the above-discussed pre-shaped delivery tools 95, 115, 135. In one embodiment where the lead 5 is pre-shaped, the lead 5 may be delivered via a standard delivery tool (e.g., stylet, guidewire, or sheath) that is substantially straight and generally does not have the same overall shape of the pre-shaped lead. Once the lead distal end 20 is secured to the implant site, the substantially straight standard delivery tool is withdrawn from the lead 5, thereby allowing the lead 5 to assume the pre-shaped configuration discussed above with respect to FIGS. 1-3B.

In one embodiment, the lead 5 is a generally standard lead that is not pre-shaped. Such lead distal end 20 may be delivered to the implantation site via standard delivery tools or pre-shaped delivery tools 95,115, 135 as described above. Once the lead distal end 20 is positioned at the implant site, a pre-shaped member, e.g., the above-described stylet 115, is inserted into and left in the implanted lead 5 to cause the lead body 25 to assume and remain in the configuration described above with respect to FIGS. 1-3B during the extent of the lead's implanted life.

Although the present invention has been described with reference to preferred embodiments, persons skilled in the art will recognize that changes may be made in form and detail without departing from the spirit and scope of the invention.

What is claimed is:

1. An implantable medical lead for implantation within a right ventricle of a heart and powered by an implantable pulse generator, the lead comprising:
a lead body including a proximal end configured to couple to the generator, a distal end, an electrode at the distal end, and a distal portion extending proximally from the distal end;
wherein, when the distal portion is in a non-deflected state, the distal portion biases to assume a configuration including first, second and third generally straight segments and first and second bends;
wherein the first segment is proximal of the distal end, the second segment is proximal of the first segment, the third segment is proximal of the second segment, the first bend is between the first and second segments, and the second bend is between the second and third segments; and
wherein, when the distal portion is implanted in the right ventricle, the configuration is at least partially the cause of the electrode being at least one of: positioned against the right ventricle septum; positioned in the outflow tract of the right ventricle; positioned for Hisian pacing; and positioned for para-Hisian pacing.

2. The lead of claim 1, wherein the first bend is defined by a first angle extending between the first and second segments of between approximately 30 degrees and approximately 70 degrees, and the second bend is defined by a second angle extending between the second and third segments of between approximately zero degrees and approximately 65 degrees.

3. The lead of claim 2, wherein the first bend has a bend radius of between approximately 1.5 cm and approximately 4 cm, and the second bend has a bend radius of between approximately 2 cm and approximately 4.25 cm.

4. The lead of claim 1, wherein the first bend exists in a first plane and the second bend exists in a second plane.

5. The lead of claim 4, wherein the first plane intersects the second plane at an angle of between approximately 60 degrees and approximately 120 degrees.

6. The lead of claim 1, wherein the first segment has a length of between approximately 1 cm and approximately 6 cm, the second segment has a length of between approximately 2 cm and approximately 5.5 cm and the third segment has a length of between approximately 23 cm and approximately 52 cm.

7. The lead of claim 1, wherein the lead body further includes a defibrillation coil extending through the first and second bends.

8. The lead of claim 1, wherein the electrode is a helical anchor.

9. The lead of claim 1, wherein, when the distal portion is implanted in the right ventricle, the configuration is at least partially the cause of the second bend being located near the apex of the right ventricle.

10. The lead of claim 9, wherein, when the distal portion is implanted in the right ventricle, the configuration is at least partially the cause of the first bend being located near the outflow tract of the right ventricle.

11. The lead of claim 1, wherein the lead body further includes a defibrillation coil extending through the first and second bends and having a length of between approximately 5.5 cm and approximately 12 cm.

12. The lead of claim 1, further comprising an insertable member extending through at least a portion of the lead and configured to cause the lead to assume the configuration when the distal portion is in a non-deflected state with the insertable member in the lead.

13. The lead of claim 12, wherein the insertable member is a pre-shaped stylet configured to be left in the lead once the lead is implanted.

14. A method of implanting an implantable medical lead in a right ventricle of a heart, the method comprising:
providing a lead body including a proximal end configured to couple to an implantable pulse generator, a distal end, an electrode at the distal end, and a distal portion extending proximally from the distal end, wherein, when the distal portion is in a non-deflected state, the distal portion biases to assume a configuration including first, second and third generally straight segments and first and second bends, wherein the first segment is proximal of the distal end, the second segment is proximal of the first segment, the third segment is proximal of the second segment, the first bend is between the first and second segments, and the second bend is between the second and third segments;
deflecting the distal portion out of its non-deflected state to deliver the distal portion into the right ventricle; and
allowing the distal portion to assume its non-deflected state within the right ventricle, wherein the configuration is at least partially the cause of the electrode being at least one of: positioned against the right ventricle septum; positioned in the outflow tract of the right ventricle; positioned for Hisian pacing; and positioned for para-Hisian pacing.

15. The method of claim 14, wherein the first bend is defined by a first angle extending between the first and second segments of between approximately 30 degrees and approximately 70 degrees, and the second bend is defined by a second angle extending between the second and third segments of between approximately zero degrees and approximately 65 degrees.

16. The method of claim 15, wherein the first bend has a bend radius of between approximately 1.5 cm and approximately 4 cm, and the second bend has a bend radius of between approximately 2 cm and approximately 4.25 cm.

17. The method of claim 14, wherein the first bend exists in a first plane and the second bend exists in a second plane.

18. The method of claim 17, wherein the first plane intersects the second plane at an angle of between approximately 60 degrees and approximately 120 degrees.

19. The method of claim 14, wherein the first segment has a length of between approximately 1 cm and approximately 6 cm, the second segment has a length of between approximately 2 cm and approximately 5.5 cm, and the third segment has a length of between approximately 23 cm and approximately 52 cm

20. The method of claim 14, wherein, when the distal portion assumes its non-deflected state in the right ventricle, the configuration is at least partially the cause of the second bend to being located near the apex of the right ventricle.

21. The method of claim 20, wherein, when the distal portion assumes its non-deflected state in the right ventricle, the configuration is at least partially the cause of the first bend to being located near the outflow tract of the right ventricle.
